(19) United States
(12) Patent Application Publication
(10) Pub. No.: US 2024/0358617 A1
(43) Pub. Date: Oct. 31, 2024

(54) Heat Stable and/or Heat Activated Biopolymers

(71) Applicant: ExoPolymer, Inc., Mountain View, CA (US)

(72) Inventors: Derek Wells, Palo Alto, CA (US); Reed A. Goodwin, San Carlos, CA (US)

(73) Assignee: ExoPolymer, Inc., Mountain View, CA (US)

(21) Appl. No.: 18/636,610

(22) Filed: Apr. 16, 2024

Related U.S. Application Data
(63) Continuation of application No. PCT/US2022/047853, filed on Oct. 26, 2022.
(60) Provisional application No. 63/271,816, filed on Oct. 26, 2021.

(51) Int. Cl. A61K 8/73; A23L 29/269; A61Q 19/10; C08B 37/00; C08J 3/075

(52) U.S. Cl. CPC A61K 8/73; A23L 29/269; A61Q 19/10; C08B 37/006; C08J 3/075; C08J 2305/00

(57) ABSTRACT

Provided herein are compositions comprising succinoglycan biopolymers lacking succinyl moieties, wherein the biopolymers are heat stable or heat activated, and methods of preparing and using such biopolymers.

### CROSS-REFERENCE TO RELATED APPLICATIONS

This application is a continuation of International Application No. PCT/US2022/047853, filed Oct. 26, 2022, which claims the benefit of priority of U.S. Provisional Application No. 63/271,816, filed Oct. 26, 2021, which is incorporated by reference herein in its entirety for any purpose.

### FIELD

Provided herein are compositions comprising succinoglycan biopolymers lacking succinyl moieties, wherein the biopolymers are heat stable and/or heat activated, and methods of preparing and using such biopolymers.

### BACKGROUND

Biopolymers, also known as biogums, polysaccharides, exopolysaccharides, or hydrocolloids, are made of chains of sugar molecules or their derivatives, linked by glycosidic bonds. In some cases, biopolymers are modified by chemical groups such as acetate or pyruvate. Although many biopolymers are derived from plants or algae, they are also produced by bacteria as exopolysaccharide (EPS). Xanthan gum, produced by Xanthomonas campestris, and gellan gum, produced by Sphingomonas elodea, are examples of microbial polymers that are produced industrially at large scales for a variety of different market applications including use in personal care formulations, food matrices, healthcare excipients, and energy production.

Industrially produced biopolymers are used as stabilizers, emulsifiers, viscosifiers, texturants, and rheological control agents. Specific examples of their unique properties include viscosity and shear-thinning in cosmetic products, stability and emulsification in packaged food, and timed release of drugs in certain pharmaceutical formulations. Other applications for biopolymers include shear-thinning fluid formulations with viscosity enhancements for the production of natural gas, prebiotic additives to food, bioremediation, and flocculation of contaminants in wastewater treatment processes. Some biopolymers have active properties, as is the case for heparin, a WHO-listed essential medicine used for blood anti-coagulation. Succinoglycan is a biopolymer produced by several microorganisms including Pseudomonas NCIB11592, Sinorhizobium meliloti, and Agrobacterium tumefaciens. It is manufactured industrially using A. tumefaciens and is commonly sold under the label Rheozan®, although it is not as commercially widespread as xanthan or gellan gums. Succinoglycan can be used in personal care, healthcare, food, and energy applications for its ability to modify the rheological properties of a solution. It is valued for its high viscosity per weight, allowing inclusion in final formulations at lower concentrations relative to xanthan or other similar biopolymers. It is also temperature stable as well as resistant to change in pH, which is useful for the formulation of acidic food products. In personal care formulations, the shear-thinning properties of succinoglycan allow for smoothness of application of final products; and in home care and cleaning products, it is valued as a stabilizer for active ingredients such as surfactants. Despite this usefulness, Rheozan® has only been approved for use in food in Japan. It is predominantly used in personal care and home care/cleaning products, and less commonly, for hydraulic fracturing.

It has been shown that when succinoglycan is heated above its transition temperature and then allowed to cool, it irreversibly loses over half of its original viscosity (Gravanis et al. 1987). For final products that require treatment at high temperatures, such as the pasteurization process, the utility of succinoglycan would therefore be limited.

### SUMMARY

The present disclosure relates, inter alia, to compositions comprising succinoglycan biopolymers lacking succinyl moieties, and wherein the biopolymers are heat stable and/or heat activated. Accordingly, the following embodiments are provided:

Embodiment 1. A composition comprising a biopolymer, wherein the biopolymer is a succinoglycan lacking succinyl moieties, and wherein the biopolymer is heat stable and/or heat activated.

Embodiment 2. The composition of embodiment 1, wherein at least 50%, at least 55%, at least 60%, at least 65%, at least 70%, at least 75%, at least 80%, at least 85%, or at least 90% of the repeating units of the biopolymer comprise a pyruvyl moiety.

Embodiment 3. The composition of embodiment 1 or embodiment 2, wherein at least 30%, at least 35%, at least 40%, at least 45%, at least 50%, at least 55%, at least 60%, at least 65%, at least 70%, at least 75%, at least 80%, at least 85%, or at least 90% of the repeating units of the biopolymer comprise an acetyl moiety.

Embodiment 4. The composition of embodiment 1 or embodiment 2, wherein less than 30%, less than 25%, less than 20%, less than 15%, less than 10%, or less than 5% of the repeating units of the biopolymer comprise an acetyl moiety.

Embodiment 5. A composition comprising a biopolymer composed of repeating units of the structure:

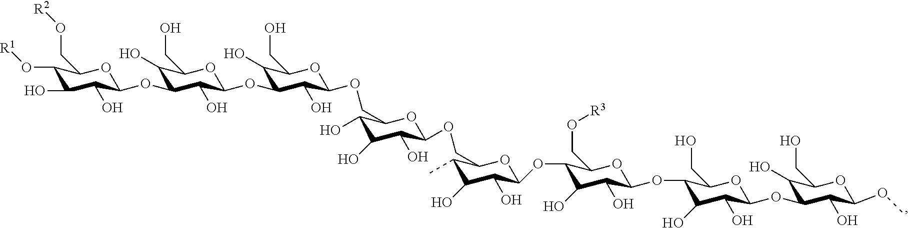

Embodiment 6. wherein the dotted lines indicate the connection between adjacent units; and

Embodiment 7. wherein for each unit, independently:
a) R³ is hydrogen or an acetyl moiety (C(O)CH₃);
b) R¹ and R² are each hydrogen, or are taken together to form a pyruvyl moiety (C(CH₃)CO₂H);

Embodiment 8. wherein at least 50%, at least 55%, at least 60%, at least 65%, at least 70%, at least 75%, at least 80%, at least 85%, or at least 90% of the units comprise a pyruvyl moiety.

Embodiment 9. The composition of embodiment 5, wherein at least 30%, at least 35%, at least 40%, at least 45%, at least 50%, at least 55%, at least 60%, at least 65%, at least 70%, at least 75%, at least 80%, at least 85%, or at least 90% of the units comprise an acetyl moiety.

Embodiment 10. The composition of embodiment 5 or embodiment 6, wherein at least at least 30%, at least 35%, at least 40%, at least 45%, at least 50%, at least 55%, at least 60%, at least 65%, at least 70%, at least 75%, at least 80%, at least 85%, or at least 90% of the units have the structure:

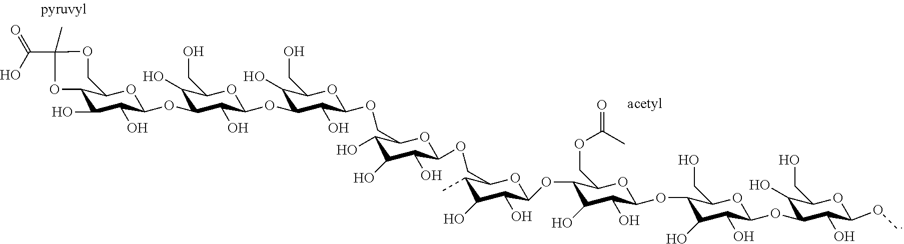

Embodiment 11. The composition of any one of embodiments 1-7, wherein the composition is heat stable and/or heat activated when heated to at least 75° C., at least 80° C., at least 85° C., at least 90° C.

Embodiment 12. The composition of embodiment 8, wherein the composition is heat stable and/or heat activated when heated to at least 75° C., at least 80° C., at least 85° C., at least 90° C. for at least 1 second, at least 5 seconds, at least 10 seconds, at least 30 seconds, at least 1 minute, at least 3 minutes, at least 5 minutes, at least 10 minutes, or at least 30 minutes.

Embodiment 13. The composition of any one of embodiments 1-9, wherein the viscosity of the composition is at least 2-fold, at least 3-fold, at least 4-fold, at least 5-fold, or at least 6-fold higher after heating to a temperature of at least 75° C., at least 80° C., at least 85° C., at least 90° C., compared to the viscosity of the composition at the same temperature prior to heating.

Embodiment 14. The composition of any one of embodiments 1-10, wherein the viscosity of the composition is at least 2-fold, at least 3-fold, at least 4-fold, at least 5-fold, or at least 6-fold higher at a second temperature after heating than at a first temperature prior to heating, wherein the first temperature and the second temperature are the same and are between 0° C.-50° C., or 0° C.-40° C., or 0° C.-30° C., or 10° C.-50° C., or 10° C.-40° C., or 10° C.-30° C.

Embodiment 15. The composition of embodiment 5, wherein less than 30%, less than 25%, less than 20%, less than 15%, less than 10%, or less than 5% of the units comprise an acetyl moiety.

Embodiment 16. The composition of embodiment 5 or embodiment 12, wherein at least at least 50%, at least 55%, at least 60%, at least 65%, at least 70%, at least 75%, at least 80%, at least 85%, or at least 90% of the units have the structure:

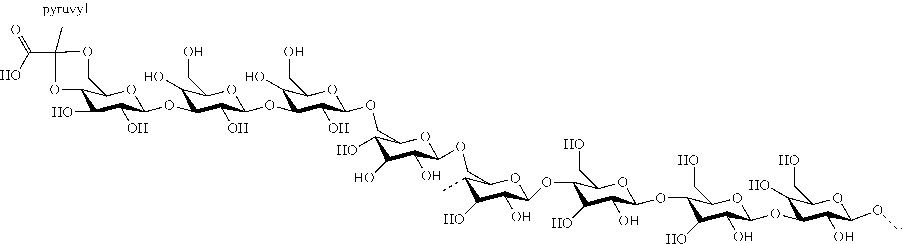

Embodiment 17. The composition of any one of embodiments 1-9, 12 and 13, wherein the composition is heat stable and/or heat activated when heated to at least 75° C., at least 80° C., at least 85° C., at least 90° C.

Embodiment 18. The composition of any one of embodiments 1-9 and 12-14, wherein the viscosity of the composition is substantially the same before and after heating to a temperature of at least 75° C., at least 80° C., at least 85° C., at least 90° C.

Embodiment 19. The composition of any one of embodiments 1-9 and 12-15, wherein the viscosity of the composition is substantially the same at a first temperature before heating and at a second temperature after heating, wherein the first temperature and the second temperature are the same and are between 0° C.-50° C., or 0° C.-40° C., or 0° C.-30° C., or 10° C.-50° C., or 10° C.-40° C., or 10° C.-30° C.

Embodiment 20. The composition of any one of embodiments 1-16, wherein the composition has been heated to at least 60° C., at least 70° C., at least 80° C., or at least 90° C. until the composition is uniformly heated and/or for at least 1 second, at least 5 seconds, at least 10 seconds, at least 30 seconds, at least 1 minute, at least 3 minutes, at least 5 minutes, at least 10 minutes, or at least 30 minutes.

Embodiment 21. The composition of any one of embodiments 1-17, wherein the composition comprises 0.01 to 1%, or 0.1 to 2%, or 0.1-10% biopolymer.

Embodiment 22. The composition of any one of embodiments 1-18, wherein the composition has been pasteurized.

Embodiment 23. The composition of embodiment 19, wherein the composition has been pasteurized by heating to a temperature of at least 80° C.

Embodiment 24. The composition of any one of embodiments 1-20, which is a water-in-oil emulsion.

Embodiment 25. The composition of any one of embodiments 1-21, wherein the composition is a food composition.

Embodiment 26. The composition of embodiment 22, wherein the food composition is a food matrix.

Embodiment 27. The composition of embodiment 22 or embodiment 23, wherein the food composition is a packaged food, shelf-stable food, pressure treated food, food that undergoes heating prior to consumption or during transit, or a sterilizable food.

Embodiment 28. The composition of any one of embodiments 22-24, wherein the food composition is a retort packaged food.

Embodiment 29. The composition of any one of embodiments 22-25, wherein the composition is a dairy product (such as ice cream, shake, processed cheese spread, cottage cheese, or yoghurt), dressing (such as high, low, or no-oil dressing), relish, sauce, soup, broth, syrup, topping, starch-based product (such as canned dessert, filling, retort pouches), dry mix product (such as dessert, gravy, beverage, sauce, or dressing), farinaceous food (such as cake), filling, confectionary, icing, frosting, meat substitute, or non-dairy beverage.

Embodiment 30. The composition of any one of embodiments 22-26, wherein the composition is a high protein liquid or non-dairy beverage, such as soy milk, almond milk, pea milk, oat milk, or rice milk.

Embodiment 31. The composition of any one of embodiments 22-26, wherein the composition is a meat substitute.

Embodiment 32. The composition of any one of embodiments 1-21, wherein the composition is a cosmetic, personal care composition, or hair care product.

Embodiment 33. The composition of embodiment 29, wherein the composition is a lotion, cream, gel, serum, liquid, cleanser, facial and/or body cleansing product, soap, cosmetic formulation, sun care product, sunscreen, tanning formulation, shampoo, conditioner, hair color, hair relaxer, spray, mousse, foam, pomade, powder, wipe, stick, or wax.

Embodiment 34. The composition of embodiment 29 or embodiment 30, wherein the composition is for application to skin or hair.

Embodiment 35. The composition of any one of embodiments 1-31, wherein the composition comprises a surfactant.

Embodiment 36. The composition of embodiment 32, wherein the composition comprises one or more of an ionic, nonionic, amphoteric, anionic, and/or zwitterionic surfactant.

Embodiment 37. The composition of any one of embodiments 1-21, 32, and 33, wherein the composition is a home care or cleaning product.

Embodiment 38. The composition of any one of embodiments 1-21, which is a drilling fluid.

Embodiment 39. The composition of embodiment 35, wherein the viscosity of the composition is substantially restored after at least one, at least two, or at least three heating and cooling cycles.

Embodiment 40. The composition of embodiment 35 or embodiment 36, wherein the composition comprises potassium and/or sodium salt.

Embodiment 41. The composition of embodiment 37, wherein the composition comprises 0.03% to 5% w/v biopolymer and a total of 5 to 120% w/v of sodium and/or potassium salt.

Embodiment 42. The composition of any one of embodiments 35-37, which is a drilling fluid with increased residence time in geological formations

Embodiment 43. The composition of any one of embodiments 1-21, which is a gravel pack slurry.

Embodiment 44. The heat stable composition of embodiment 40, wherein the gravel pack slurry comprises sand and water.

Embodiment 45. The heat stable composition of embodiment 41, wherein the biopolymer is present in an amount sufficient to maintain the sand in suspension in the water at 0° C.-50° C., or 0° C.-40° C., or 0° C.-30° C., or 10° C.-50° C., or 10° C.-40° C., or 10° C.-30° C.

Embodiment 46. A method comprising heating the composition of any one of embodiments 1-42.

Embodiment 47. The method of embodiment 43, wherein the method comprises heating the composition to at least 75° C., at least 80° C., at least 85° C., at least 90° C. until the composition is uniformly heated and/or for at least 1 second, 5 seconds, at least 10 seconds, at least 30 seconds, at least 1 minute, at least 3 minutes, at least 5 minutes, at least 10 minutes, or at least 30 minutes.

Embodiment 48. The method of embodiment 44, wherein after heating, the method comprises cooling the composition to a temperature between 0° C.-50° C., or 0° C.-40° C., or 0° C.-30° C., or 10° C.-50° C., or 10° C.-40° C., or 10° C.-30° C.

Embodiment 49. The method of embodiment 45, comprising more than one heating and cooling cycle.

Embodiment 50. The method of any one of embodiments 43-46, wherein the viscosity of the composition is substantially the same after cooling as it was at the same temperature before heating.

Embodiment 51. The method of any one of embodiments 43-46, wherein the viscosity of the composition is at least 2-fold, at least 3-fold, at least 4-fold, at least 5-fold, or at least 6-fold higher after cooling than at the same temperature before heating.

Embodiment 52. A method comprising applying pressure 100 MPa to the composition of any one of embodiments 1-42.

Embodiment 53. The method of embodiment 49, wherein the viscosity of the composition is either substantially the same after application of pressure as it was before application of pressure, or the viscosity of the composition is at least 2-fold, at least 3-fold, at least 4-fold, at least 5-fold, or at least 6-fold higher after application of pressure than before application of pressure.

Embodiment 54. A method of preparing a composition comprising incorporating the composition of any one of embodiments 1-21 into a food or beverage.

Embodiment 55. A method of preparing a composition comprising incorporating the composition of any one of embodiments 1-21 into a cosmetic, personal care composition, or hair care product.

Embodiment 56. The method of embodiment 51 or 52, further comprising heating the composition.

Embodiment 57. The method of embodiment 53, wherein the method comprises heating the composition to at least 75° C., at least 80° C., at least 85° C., at least 90° C. until the composition is uniformly heated and/or for at least 1 second, at least 5 seconds, at least 10 seconds, at least 1 minute, at least 3 minutes, at least 5 minutes, at least 10 minutes, or at least 30 minutes.

Embodiment 58. The method of embodiment 53 or 54, wherein after heating, the method comprises cooling the composition to a temperature between 0° C.-50° C., or 0° C.-40° C., or 0° C.-30° C., or 10° C.-50° C., or 10° C.-40° C., or 10° C.-30° C.

Embodiment 59. The method of embodiment 55, comprising more than one heating and cooling cycle.

Embodiment 60. The method of any one of embodiments 53-56 wherein after heating, the viscosity of the composition is either substantially the same after cooling as it was at the same temperature before heating, or the viscosity of the composition is at least 2-fold, at least 3-fold, at least 4-fold, at least 5-fold, or at least 6-fold higher after cooling than at the same temperature before heating.

Embodiment 61. The method of any one of embodiments 51-57, further comprising applying pressure 100 MPa to the composition.

Embodiment 62. The method of embodiment 58, wherein the viscosity of the composition is either substantially the same after application of pressure as it was before application of pressure, or the viscosity of the composition is at least 2-fold, at least 3-fold, at least 4-fold, at least 5-fold, or at least 6-fold higher after application of pressure than before application of pressure.

Embodiment 63. A method of maintaining or increasing the stability of a product exposed to at least one heating and cooling cycle comprising incorporating a composition of any one of embodiments 1-21 into the product.

Embodiment 64. The method of embodiment 60, wherein the heating and cooling cycle comprises heating the product to at least 75° C., at least 80° C., at least 85° C., at least 90° C. until the composition is uniformly heated and/or for at least 1 second, at least 5 seconds, at least 10 seconds, at least 30 seconds, at least 1 minute, at least 3 minutes, at least 5 minutes, at least 10 minutes or at least 30 minutes, and after heating, cooling the product to a temperature between 0° C.-50° C., or 0° C.-40° C., or 0° C.-30° C., or 10° C.-50° C., or 10° C.-40° C., or 10° C.-30° C.

Embodiment 65. The method of embodiment 60 or 61, wherein after heating, the viscosity of the product is either substantially the same after cooling as it was at the same temperature before heating, or the viscosity of the product is at least 2-fold, at least 3-fold, at least 4-fold, at least 5-fold, or at least 6-fold higher after cooling than at the same temperature before heating.

Embodiment 66. A method of indicating temperature fluctuation during transit or storage of a product comprising incorporating a composition of at least one of embodiments 1-21 into the product before transit or storage, and evaluating viscosity of the product after transit or storage.

Embodiment 67. The composition of any one of embodiments 22-28, wherein the composition is manufactured by incorporating the composition of any one of embodiments 1-21 into a food or beverage.

Embodiment 68. The composition of any one of embodiments 29-31, wherein the composition is manufactured by incorporating the composition of any one of embodiments 1-21 into a cosmetic, personal care composition, or hair care product.

Embodiment 69. The composition of embodiment 25, wherein the retort packaged food is manufactured by incorporating the composition of any one of embodiments 1-21 into a food or beverage, incorporating the food composition into a package, optionally sealing the package, heating the package to 110-135° C., and subsequently cooling the package.

Embodiment 70. The composition of embodiment 66, wherein after heating, the viscosity of the composition is either substantially the same after cooling as it was at the same temperature before heating, or the viscosity of the composition is at least 2-fold, at least 3-fold, at least 4-fold, at least 5-fold, or at least 6-fold higher after cooling than at the same temperature before heating.

Embodiment 71. The composition of any one of embodiments 1-21 for use in food preparation or packaging.

Embodiment 72. The composition of any one of embodiments 1-21 for use in cosmetic, personal care composition, or hair care products.

### BRIEF DESCRIPTION OF THE DRAWINGS

FIG. 1. Structure of succinoglycans. Source organism and relevant genotypes are shown at right.

FIG. 2. Succinoglycan biosynthetic gene clusters and arrangement in S. meliloti and A. tumefaciens. The exs genes are shown in solid black while exo genes are outlined. In S. meliloti all dedicated succinoglycan biosynthetic and structural genes are located in a single cluster. In A. tumefaciens several clusters are scattered across the linear chromosome.

FIG. 3. Recovery of solution viscosity after heating. 1 mL of a 0.5% Rheozan® or EXO4 solution was heated to 80° C. for 5 minutes and allowed to cool. A, flow curves of Rheozan® and EXO4 collected from 5×10⁻³ s⁻¹ to 5×10⁴ s⁻¹ at 20° C. B, curves were fit with the Cross model and η₀ was used to calculate the percent viscosity remaining after heating for Rheozan®, EXO4, and EXO5.

FIG. 4. Temperature ramps of 0.5% Rheozan® (A) and 0.5% EXO4 (B) collected at a constant shear rate of 1 s⁻¹. Samples were heated from 20° C. to 85° C. (solid lines) at 2° C. min⁻¹ then cooled back to 20° C. (dashed lines) at the same rate.

FIG. 5. Recovery of EXO5 viscosity after heating. Temperature ramp performed at 1 s⁻¹ heating from 20° C. to 85° C. at a rate of 2° C. min⁻¹ (solid line) then cooled to 20° C. (dashed line).

FIG. 6. Viscosity gain of EXO5 after heating for the indicated amount of time at 80° C. After samples cooled flow sweeps were collected and the data fit with the Cross equation. Values represent the percent zero-shear viscosity relative to unheated sample.

FIG. 7. Gel formation of high concentration EXO5. Solutions of 1.5% (w/v) were autoclaved and allowed to cool to room temperature (vial 2), and compared to an unheated sample (vial 1). Heat-treated EXO5 (vial 2) formed an immobile, weak gel, in comparison to the unheated sample (vial 1). See (A). Inverted vials (B) showed that the heat treated EXO5 sample formed a gel at the bottom of the tube.

### DETAILED DESCRIPTION

### Biopolymers and Biopolymer Preparations

Species belonging to the Rhizobiaceae, a family of soil-dwelling bacteria, have been studied for decades for their ability to provide fixed nitrogen to their leguminous plant hosts, but to date have not generally been exploited as fermentative microorganisms for the production of bioindustrial, pharmaceutical, or cosmetic products. These bacteria naturally produce water-soluble exopolysaccharides, or biopolymers, which have roles in both host plant association and biofilm formation. The variety of exopolysaccharides produced by the Rhizobiaceae suggests a breadth of novel biopolymers with new functionalities that could add substantial value to several markets.

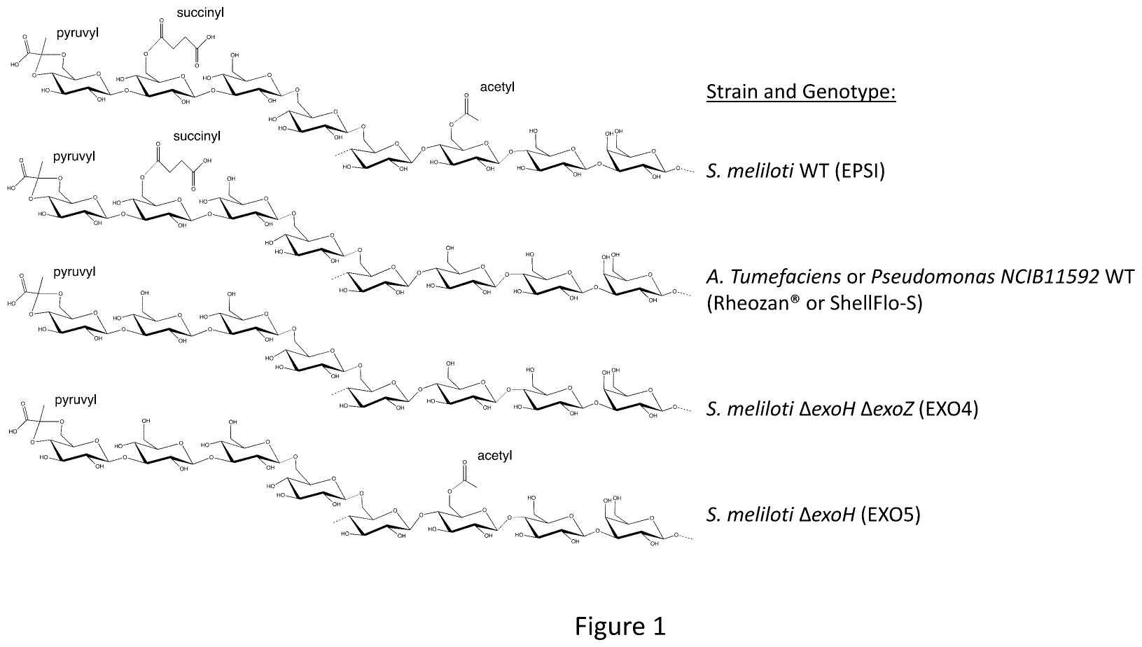

Succinoglycan Sinorhizobium (Ensifer) meliloti naturally produces two acidic exopolysaccharides: succinoglycan (EPS I) and galactoglucan (EPS II) (Barnett 2018). The repeating unit of succinoglycan (FIG. 1) consists of glucose and galactose in a 7:1 ratio with acetyl, pyruvyl, and succinyl modifications (Reuber 1993). Succinoglycan is naturally produced by S. meliloti at both high (on the order of 10⁶ Da) and low molecular weights. The low molecular weight fraction is comprised of monomers, dimers, and trimers (Gonzalez 1998).

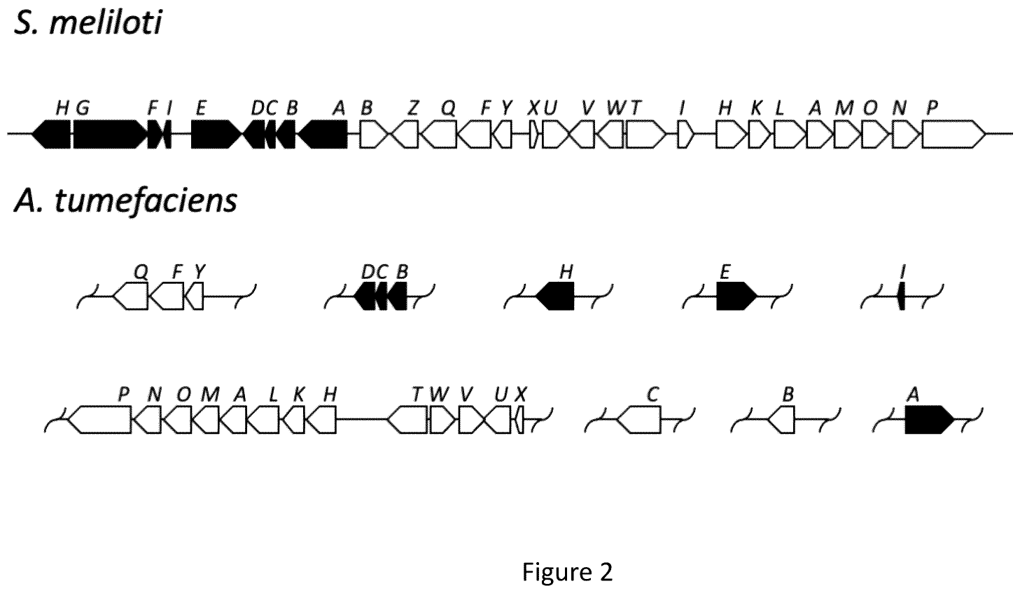

Agrobacterium and Pseudomonas species secrete a succinoglycan molecule that differs from that produced by S. meliloti in that it lacks an acetyl group (FIG. 1) (Chouly 1995). The exs-exo clusters of both bacteria encode the same set of genes with the exception of exoZ, encoding the acetyltransferase, which is absent from Agrobacterium gene clusters (FIG. 2). While Agrobacterium species only produce a high molecular weight succinoglycan, the gene cluster encodes both the ExoK and ExsH glycanases. Although glycanases are generally considered to generate low molecular weight succinoglycan from S. meliloti, their presence in the Agrobacterium genome suggests they may also determine the size distribution of high molecular weight succinoglycan. Additionally, a rearrangement appears to have occurred as these two genera diverged leading to differences in gene order and intergenic regions. It has also been shown that deletion of exoK or exsH eliminates the production of LMW succinoglycan in S. meliloti. S. meliloti strains harboring mutations in these genes produce HMW, and therefore higher viscosity, succinoglycan.

### Strain Construction

S. meliloti is amenable to genetic modification, and a common method for strain engineering is to use homologous recombination, antibiotic resistance, and sucrose counter selection (Quandt 1993) to delete specific regions in the genome. Plasmids that contain modified genomic regions can be constructed and used to replace native regions with targeted changes. By introducing these non-replicating plasmids by conjugal transfer, strains with single integrations can be selected by antibiotic resistance and confirmed by PCR. Integrated plasmids can then be counter selected due the presence of the sacB gene, which encodes a levansucrase that is lethal to Gram negative bacteria in the presence of sucrose. Antibiotic sensitive, sucrose resistant strains will then either have recombined to wild type, or have incorporated a deletion, insertion, or other modification that was present in the constructed plasmid. Modified strains can be confirmed by PCR and sequencing.

Unmodified, non-domesticated strains of S. meliloti produce both succinoglycan and galactoglucan, and are suitable for the simultaneous production of both biopolymers. In certain type strains, such as Rm1021 (ATCC51124), the ability to produce galactoglucan has been lost during lab strain domestication (Charoenpanich 2015). For the production of succinoglycan in the absence of galactoglucan, the type strain Rm1021 can be used. There are several regulatory genes that can be modified, resulting in strains that overproduce succinoglycan. These genes include exoR, exoS, chvl, syrM, and nodD3 (Barnett 2015). Others include syrA, mucR (Keller 1995), and exoX (Zhan 1990). If a non-domesticated strain of S. meliloti is used, in some embodiments, galactoglucan biosynthetic genes may be knocked out to generate a strain that only produces succinoglycan. The genes required for galactoglucan biosynthesis fall within a 32 kb region of pSymB and include six predicted glycosyltransferases and four genes predicted to encode proteins required for the synthesis of dTDP-glucose and dTDP-rhamnose (Becker 1997). Any of several glycosyltransferases, such as wgaB or wgeB may be excised to eliminate production of galactoglucan.

S. meliloti produces succinoglycan as a mixture of high and low molecular weight forms. The low molecular weight form is important for recognition by host plants during symbiosis. During synthesis the nascent succinoglycan chain is cleaved by the glycanases ExoK and ExsH to generate the low molecular weight fraction (York 1998). These glycanases require the succinyl group to recognize their substrate and only cleave succinoglycan that is being actively synthesized (i.e., neither protein is active against purified succinoglycan). Deleting either the exoK or exsH gene increases the ratio of high:low molecular weight succinoglycan, with exsH mutants exhibiting a larger shift in the ratio. Deletion or inactivation of both genes eliminates most or substantially all of the low molecular weight succinoglycan.

### Methods of Making Biopolymers

For production of biopolymers, various liquid growth media can be used. S. meliloti strains grow well on LB or TY medium, and these can be supplemented with additional carbon sources such as glucose, sucrose, and/or succinate to boost production of product. S. fredii can be grown on TY medium, and supplementation with additional carbon source is beneficial to production. Both S. meliloti and S.fredii can be grown on defined minimal medium, such as M9 or MOPS-mannitol, which, in some instances, can result in higher yields of exopolysaccharide product. Minimal medium allows for improved control over fermentation variables such as phosphate concentration, pH, micronutrients, sulfate concentration, and carbon source.

Alcohol precipitation may be used to purify biopolymers after fermentation. Typically, cells are removed from fermentation broth by centrifugation or filtration. In some instances, the entire fermentation broth is precipitated, and cell debris remains in the biopolymer preparation. In some embodiments, if the fermentation broth has high viscosity, one to two volumes of water may be added to assist in cell separation procedures. To further remove residual cells or cell debris, the cell-free supernatant may be incubated with protease. In some embodiments, to precipitate biopolymer, isopropanol or ethanol, as well as a mono- or divalent cation such as KCl or CaCl₂ in a concentration range around 1 mM, can be added to the cell-free supernatant, typically at 1× to 2× the culture volume. Biopolymers may precipitate upon mixing, and can be isolated by centrifugation or filtration. Further purification steps may be performed to reduce salt concentrations and/or remove cell debris that may have precipitated with the polymer. These steps may include additional solvent washes, protease treatments, dialysis, etc., according to the desired end use. Purified product can be dried in an oven until the mass stabilizes (e.g., until all unbound water has evaporated), or lyophilized. Dried product can be ground, milled, or otherwise processed.

Thermostable (Heat Stable and/or Heat Activated) Biopolymers Suitable for Heat Treatment

Purified biopolymers can be formulated into different compositions including compositions for personal care, healthcare, food, and energy applications. In certain instances, such as in personal care, healthcare, or food formulations, sterilization (pasteurization) may be used to prevent contamination and ensure quality, performance, and safety. In other instances, such as for the production of energy, it would be desirable for biopolymers to maintain rheological performance under or after exposure to high temperatures. Thus, in some instances, biopolymers may be treated at up to 80° C. or higher for several minutes, and then cooled for use in a given application. Final formulations with ingredients that are resistant to high temperature may also be heat treated.

The ability to recover viscosity after heating can be beneficial, for example, where the biopolymer is used as a viscosity modifier or stabilizer. Thermotolerant (heat stable and/or heat activated) biopolymers can be used at lower concentrations in pasteurized products as no correction in formulation needs to be made for loss of viscosity post heating. This property would be of particular value in food and personal care formulations to maintain product quality, stability, and safety during manufacturing. Heat tolerant biopolymers could additionally be useful in formulations where heating is necessary for final product quality or performance. Examples are emulsions that require heating for proper formation, or active ingredients that are only soluble at high temperature.

Many shelf stable foods and other products are prepared by high-pressure or ultra-high-pressure treatment. This process utilizes high pressure with low to mid-levels of heat (usually less than 45C) primarily to disrupt bacterial and fungal cell walls. The pressure level applied to the food is typically ≥100 MPa, for example between 250-600 MPa. The shear-thinning properties of heat stable or heat activated biopolymers may be beneficial for this application. Products that are processed in this manner would maintain low viscosity during processing, and then desirable end viscosities would be attained upon final packaging.

Biopolymers are also used in oil wells both as a drilling and gravel pack fluids. While succinoglycans derived from A. tumefaciens have been found to perform well for these purposes, high temperatures permanently reduce the viscosity of the fluid, thereby decreasing its recyclability. The use of a heat stable and/or heat activated biopolymer in drilling and packing fluids would allow for increased recovery and decreased operating costs and extended well productivity.

Biopolymers with Heat-Activated Viscosity Increase

Biopolymers with heat-activated viscosity increase may be useful in a number of different applications where products must undergo a heating or cooking step prior to achieving final material properties and performance. Specifically, this could include food products that are cooked or personal care products that contain ingredients that require heating for proper function.

Food pasteurization or emulsion formation and stabilization are further examples.

The ability to manipulate a less viscous fluid, prior to heating, is another advantage of heat activated viscosity increase. Biopolymers are often incorporated into dairy products such as yogurts and cheeses to provide texture and stability. The milk used to prepare these products is typically vat pasteurized before fermentation to eliminate spoilage organisms. Incorporation of biopolymers pre-pasteurization would be desirable to reduce the possibility of contamination from ingredients added later. Biopolymers that are heat-activated would be particularly advantaged for this use; they can be added at a low concentration, facilitating liquid handling due to reduced viscosity, and then final viscosity of the food product would be achieved post-heating. In some embodiments, a “heat-activated biopolymer” is a biopolymer whose viscosity increases following heating.

Heat stable and/or heat activated biopolymers could be particularly beneficial in packaged food that requires sterilization, for example, in retort treatment and retort packaged food. Many shelf-stable foods and other products are prepared by retort treatment—sterilization under high temperature and pressure, similar to autoclaving. High viscosity however reduces heat transfer, posing a problem for retort efficiency in thick products. The ability to recover high levels of viscosity or increased viscosity after heating (i.e., heat induced thickening) would be beneficial for the preparation of shelf-stable foods and other products where heat sterilization is required. Thus, a food product with low initial viscosity which thickens to its desired viscosity after heat sterilization would allow for a more efficient sterilization process. In some embodiments, a retort packaged food can be manufactured by incorporating the biopolymers as discussed herein into a food or beverage, incorporating the composition into a package (such as a pouch, tray, or bottle using plastic materials or a flexible metal-plastic laminate or metal can), optionally sealing the package, heating the package to high temperatures (typically from 110-135° C.), and subsequently cooling the package, wherein the viscosity of the composition is either substantially the same after cooling as it was at the same temperature before it was heated and cooled, or wherein the viscosity of the composition is increased (i.e., at least 2-fold, at least 3-fold, at least 4-fold, at least 5-fold, or at least 6-fold higher) after cooling compared to the same temperature before heating.

Biopolymers that change viscosity after a heating and cooling cycle may have properties that are beneficial when used alone or when added to a number of products across a wide range of markets. For example, there may be benefit of delivering a low viscosity product to the end consumer which then increases viscosity after a heating and cooling cycle. This could be advantageous in cooking sauces and soups that cool to a desired consistency after heating. Further, in personal care applications, there may be a benefit to delivering a low viscosity product to the consumer which then increases viscosity after a heating and cooling cycle. Hair care products in particular may benefit from this type of performance, especially those that are applied to the hair and then blow-dried to increase volume. For example, a product including biopolymers of the present disclosure can be used as a volumizing agent for hair styling, by applying to wet or dry hair and heating by blow-drying, followed by cooling, to result in a hair style with increased volume. In some embodiments, the biopolymer discussed herein can be incorporated in hair styling products, such as hair oil, hair cream, hair wax, hair spray, hair serum, hair paste, hair mousse, hair gel, hair powder, etc. The ability to stabilize or reduce the use of other ingredients in hair styling products, such as surfactants, may also be beneficial. Increased stabilization upon heating may allow for reduced levels of petrochemical ingredients in consumer products, and potentially reduce product volumes, subsequent packaging and transportation costs, and energy inputs.

Heat stable and/or heat activated biopolymers could also be particularly beneficial as ingredients in industries in which large volumes of liquid must be moved or pumped from one location to another. In particular, the ability to recover high levels of viscosity or increased viscosity after heating may allow flexibility during drilling applications (e.g., the ability to pump low viscosity heated material into formations which then becomes more viscous and stable in situ).

Heat stable and/or heat activated biopolymers may also increase shelf stability, cold chain disruption resistance, and product quality. Biopolymer ingredients that change viscosity as a result of exposure to temperature fluctuations, such as a heating/cooling cycle or multiple temperature cycles, can confer improved stability when incorporated in products. In particular, if refrigeration is lost during transit or storage of products that require refrigeration (due to power loss, for example), a heat stable or activated biopolymer ingredient may provide increased resistance to product breakdown or loss. Products containing biopolymers of the present disclosure would thus have extended shelf life over products lacking such stabilizing agents, when subjected to temperature fluctuations. Another use of biopolymers of the present disclosure would be as indicators or markers of temperature fluctuations during transit or storage. Products with substantially increased viscosities would indicate inconsistencies in transit or handling.

### Compositions

Heat stable and/or heat activated biopolymers may be used in a variety of compositions. These include, but are not limited to, cosmetic and personal care compositions, hair care products, detergents, flowable pesticides, liquid feed supplements, cleaners, abrasives, polishers, ceramics, foundry coatings, texturized coatings, slurry explosives, dye and pigment suspensions, paints, creams, lotions, pharmaceuticals, home and household cleaning compositions, oil field chemicals, drilling fluids (muds), workover and completion fluids, stimulation and enhanced oil recovery, foods, beverages, and food and beverage compositions.

Cosmetic and personal care compositions containing heat stable and/or heat activated biopolymers may include, but are not limited to, skin lotions and creams, gels, serums and liquids, facial and body cleansing products (e.g., body wash), anti-acne products, wipes, liquid and bar soap, color cosmetic formulations, make-ups, foundations, sun care products, sunscreens, tanning formulations, shampoos, conditioners, hair color formulations, hair relaxers, products with alpha- or beta-hydroxy acids, and hair fixatives such as sprays, gels, mousses, pomades, and waxes, including low VOC hair fixatives. The compositions may be in any form, including without limitation, emulsions, gels, liquids, sprays, solids, foams, mousses, powders, wipes, or sticks.

Food and beverage formulations containing heat stable and/or heat activated biopolymers include, but are not limited to, dressings, relishes and sauces, soups, broths, syrups and toppings, starch-based products, canned desserts, fillings, dry mix products (desserts, gravies, beverages, sauces, dressings), farinaceous foods, dairy products (ice cream, yogurt, shakes, processed cheese spread, cottage cheese), and confectionary. Also included are alternative protein foods such as plant-based sausages, patties, or any other non-animal product that aims to mimic the texture, taste, nutrient content, and mouthfeel of meat. Other alternative protein products are non-dairy beverages such as soy-, almond-, rice-, pea-, or oat-milks and substitute cheeses. Heat stable and/or heat activated biopolymers may also be used in gluten-free products such as baked goods including bread, pastries, and desserts.

Home care and cleaning formulations containing heat stable and/or heat activated biopolymers include, but are not limited to, products that contain surfactants, either non-ionic, anionic, cationic, zwitterionic, or amphoteric. Other cleaning formulations containing heat stable and/or heat activated biopolymers are products that contain bases, acids, bleaches, detergents, abrasives, sanitizers (such as alcohols), antimicrobial agents, and spirit solvents. The compositions may be in any form, including without limitation, emulsions, gels, liquids, sprays, solids, foams, mousses, or powders.

Heat stable and/or heat activated biopolymers may be formulated into compositions in any amount that provides the desired properties. Typically, less heat stable biopolymer will be needed to achieve the same properties and functionality as native, unmodified succinoglycans such as Rheozan®. The heat stable and/or heat activated biopolymers will typically be used in an amount of at least about 0.01%, particularly at least about 0.5%, more particularly at least about 0.75% and less than about 20%, particularly less than about 15%, more particularly less than about 10%, or less than about 5%. In some embodiments, the heat stable and/or heat activated biopolymers are used in an amount from 0.01% to 10%, or an amount between 0.1% and 5%, or an amount between 0.1% and 3%. The amount used will depend not only upon the properties desired, but also upon the intended use and desired viscosity, as well as the nature of other ingredients.

### Examples

### Example 1. Succinoglycan Produced by S. meliloti

Succinoglycan, also referred to as EPS I, is naturally produced by Sinorhizobium meliloti (FIG. 1). The repeating unit consists of a linear main chain of one galactose and three glucose monosaccharides, and a side chain of four glucose molecules. Main chain sugars are linked by β-1,3 and β-1,4 glycosidic bonds. Side chain sugars are linked by β-1,3 and b β-1,6 glycosidic bonds. It is acetylated on glucose three of the main chain, succinylated on glucose three of the side chain, and pyruvylated on the terminal glucose of the side chain (Reuber 1993).

### Example 2. Strain Construction

For targeted deletion of selected ORFs, excision of insertion elements, or correction of SNPs, a non-replicating plasmid vector with positive and negative selection markers was used.

Since S. meliloti is amenable to genetic modification using standard molecular biology and strain engineering techniques, this methodology allows for rapid and precise changes to their genomes to create desired genotypes. First, derivatives of the pJQ200SK plasmid (Quandt 1993) carrying deletion cassettes were generated. For deletion cassettes, regions upstream and downstream (usually 500 bp) of the target ORF including start and stop codons were amplified by PCR. For introduction of wild type DNA, regions upstream and downstream of an insertion element were amplified by PCR. Next, plasmids were assembled using the CPEC method (Quan 2009), and sequence verified prior to introduction into S. meliloti. Plasmids were introduced into S. meliloti by tri-parental mating and strains containing single integrations at homologous genomic regions were selected for antibiotic resistance and verified by PCR using primers outside of amplified regions. Strains positive for integration of plasmids were then streaked to purification and selected for the ability to grow on sucrose. The presence of the sacB gene on the integrated pJQ200 plasmid causes lethality when strains are grown on sucrose. Strains that are propagated on sucrose will therefore have mutations in sacB itself or will recombine to “loop out” the integrated plasmid and either revert to wild type or harbor the deleted or modified sequence originally present in the plasmid.

The targeted deletion method described above can be used to generate strains that produce individual biopolymers or those that lack chemical modifications. In S. meliloti strains that only produce succinoglycan, genes responsible for succinylation and acetylation, exoH and exoZ, for example, may be deleted. A strain producing succinoglycan lacking the acetyl group can be built by deleting the acetyltransferase encoding gene exoZ. Strains with deletions in the succinyltransferase encoding gene exoH produce succinoglycan without the succinyl group. Strains deleted for either of the glycanase encoding genes exoK and exsH produce more high molecular weight succinoglycan, while a strain carrying both deletions does not produce low molecular weight succinoglycan. Strains with single deletions were constructed as above resulting in RAG195 (ΔexoZ), RAG225 (ΔexoH), RAG236 (ΔexoK), and RAG237 (ΔexsH). The exoH and exoK open reading frames only have a few bp between them, allowing the genes to be deleted in tandem to produce strain RAG282. The ΔexoKΔexsH double glycanase mutant RAG248 was built by transducing the exsH pJQ200 insertion into RAG236 then selecting for sucrose-resistant colonies with deletions in both genes. The ΔexoHΔexoZ strain RAG272 was built by deleting exoZ from RAG225. Strain RAG283, which is ΔexoHKΔexsH was built by deleting exoHK from RAG237. The ΔexoHΔexsH strain RAG285 was built by transducing the exsH insertion into RAG225 and selecting for sucrose resistant colonies that were deleted for both genes.

### Example 3. Production and Purification of Biopolymers

Bench scale biopolymer production was performed in shake flasks. Strains were inoculated into a buffered medium containing 1% glucose and grown overnight at 30° C. The next day, the overnight cultures were diluted, typically at a ratio of 1:50, into production medium. Production medium consisted of a defined minimal medium such as M9 containing a carbon source, either glucose or sucrose, at a concentration between 2-4% (w/v), a nitrogen source such as ammonium sulfate, a buffer to maintain neutral pH, divalent cations such as MgSO₄ and CaCl₂, trace elements, and vitamins (U.S. Pat. No. 73,715,581B2). Strains were grown in production medium for up to three days, and then harvested for purification.

Recovery and purification of biopolymers were performed by initial cell separation followed by alcohol precipitation. Cultures were diluted in either two or three volumes of water and supernatant was separated from cells by centrifugation. Supernatants were then decanted and treated with proteinase K (VWR) and held at 50° C. overnight to remove any residual protein or cell debris. Biopolymers were precipitated at room temperature by addition of two volumes of isopropyl alcohol. Precipitates were collected on filter paper using suction, then washed with isopropyl alcohol followed by 70% ethyl alcohol. The material was dried at 60° C. until the weight stabilized, typically overnight. The final product was ground with a mortar and pestle or impact milling.

TABLE 1
Strains

| Strain | Relevant Genotype | Polymer modification |
|---|---|---|
| EXO1 | ΔwgeB | none |
| RAG195 | ΔwgeBΔexoZ | Unacetylated polymer |
| RAG225 | ΔwgeBΔexoH | Unsuccinylated polymer |
| RAG236 | ΔwgeBΔexoK | Polymer with increased |
| | | molecular weight |
| | | distribution |
| RAG237 | ΔwgeBΔexsH | Polymer with increased |
| | | molecular weight |
| | | distribution |
| RAG248 | ΔwgeBΔexoKΔexsH | Polymer with increased |
| | | molecular weight |
| | | distribution |
| RAG272 | ΔwgeB ΔexoHΔexoZ | Unsuccinylated, |
| | | unacetylated |
| RAG282 | ΔwgeB ΔexoHΔexoK | Unsuccinylated polymer |
| | | with increased |
| | | molecular weight |
| RAG283 | ΔwgeB ΔexoHΔexsH | Unsuccinylated polymer |
| | | with increased |
| | | molecular weight |
| RAG285 | ΔwgeB ΔexoHΔexoKΔexsH | Unsuccinylated polymer |
| | | with increased molecular |
| | | weight |

TABLE 2
Table of succinoglycans and derivatives

| Molecule | Strain | Acetyl | Succinyl | Pyruvyl |
|---|---|---|---|---|
| Succinoglycan | EXO1 | X | X | X |
| (S. meliloti Rm1021) | | | | |
| Rheozan ®, ShellFlo-S | RAG195 | | X | X |
| EXO4 | RAG272 | | | X |
| EXO5 | RAG225 | X | | X |

### Example 4. Heat Treatment and Rheological Characterization

Polymers were dissolved in 1% NaCl with rapid stirring, then allowed to fully hydrate overnight at 4° C. For comparison a solution of a commercially sourced Rheozan® SH (Solvay) was prepared identically. The flow properties of purified polymers were measured using a DHR3 rheometer (TA Instruments) with a 40 mm 200 cone geometry and a Peltier plate set to 20° C. For flow sweeps the polymer viscosity was measured as a function of shear rate from 5×10⁻⁴ s⁻¹ to 5×10³ s⁻¹. Zero-shear viscosity was determined by fitting data to the Cross equation:

$$\frac{η - {η}_{0}}{{η}_{0} - {η}_{∞}} = \frac{1}{1 + {( K \dot{γ} )}^{m}}$$

Where η₀ is the zero-shear viscosity; η_{∞} is the infinite shear viscosity; γ is the strain rate; K is the cross constant; m is the shear thinning index.

Two methods were employed to test viscosity recovery after heating. For temperature ramps the geometry edge was sealed with mineral oil to prevent evaporation. Viscosity was measured at a constant shear rate of 1 s⁻¹ while increasing the temperature from 20° C. to 85° C. at a rate of 2° C. min⁻¹, then cooled to 20° C. at the same rate.

To confirm the results from temperature ramps 1 mL of each solution was heated 5 min at 80° C. in a sealed tube. Tubes were cooled and mixed well. Flow curves were generated for both heated and unheated samples drawn from the same stock solution as described above.

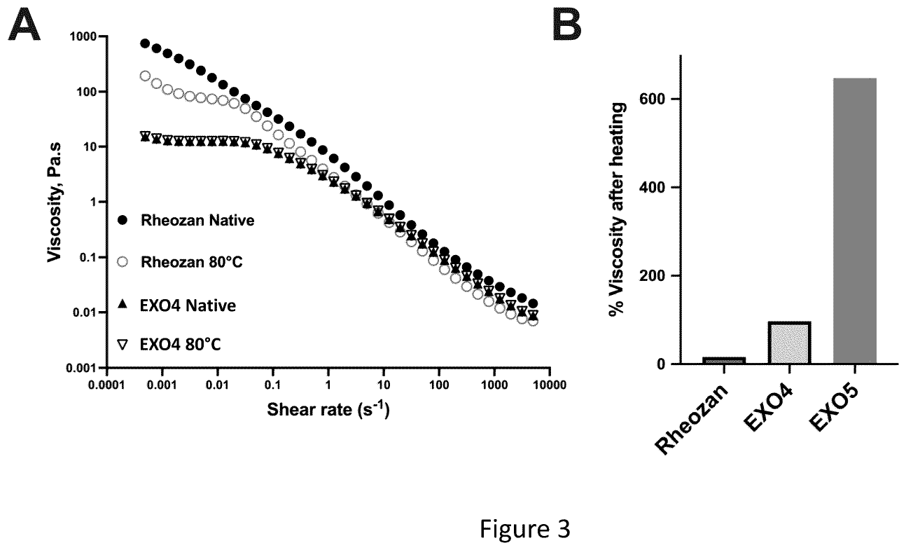

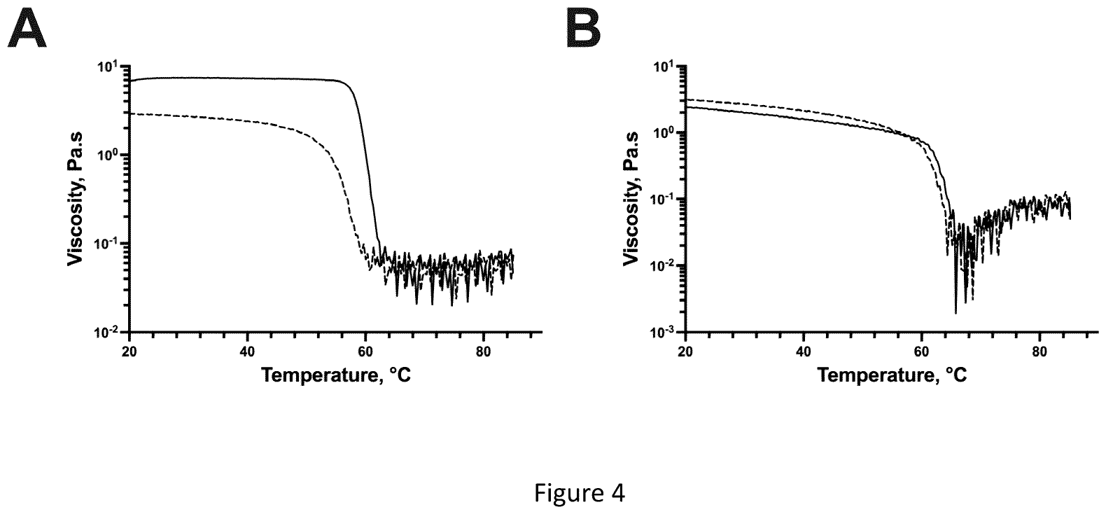

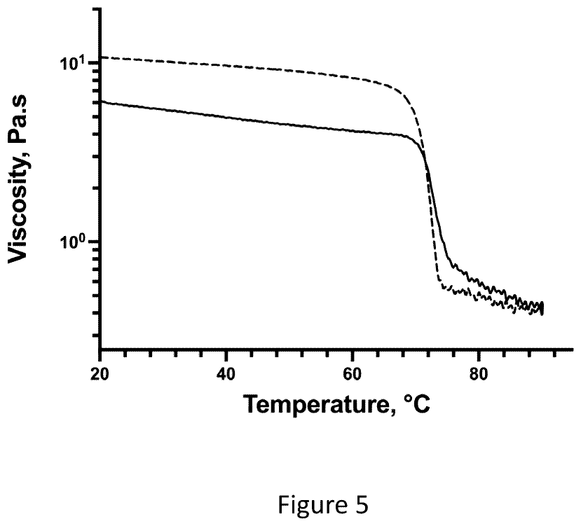

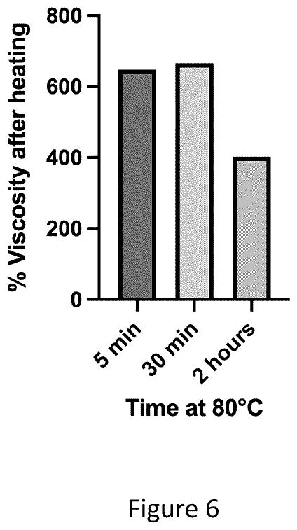

Regardless of the method used, Rheozan® exhibited a permanent loss of viscosity following heat treatment (FIG. 3 and FIG. 4). Results show that after heating Rheozan® for 5 min at 80° C. this solution retained only 16% of the zero-shear viscosity as the untreated sample. In contrast, heating EXO4 does not cause a permanent loss of viscosity. Under the same conditions the heated and cooled EXO4 solution had over 97% of the zero-shear viscosity as the untreated solution, as shown in FIG. 3B. When an EXO5 solution was heated above its melting temperature then cooled it increased viscosity relative to the native solution. FIG. 5 shows the temperature ramp data for EXO5. Solutions of EXO5 that were heated to 80° C. for 5 min, 30 min, or 2 h recover 648%, 665%, or 402% of their viscosity, respectively, as shown in FIG. 3B (5 min treatment) and FIG. 6.

### Example 5. Pressure Treatment and Rheological Characterization

In further experiments, solutions containing biopolymers of the present disclosure are subjected to high pressure to measure flow rates. The pressure level applied is typically ≥100 MPa, for example between 250-600 MPa. Biopolymer solutions with shear thinning properties, where viscosity decreases as shear rate increases (as shown in FIG. 3A), display high flow rates and are thus amenable to pressure treatment. Pressure treated biopolymer solutions may also be subjected to heating to provide suitable final viscosities for end products.

### Example 6. Stability of EXO5

To examine the durability of the heat-activated viscosity increase, the resistance of EXO5 to multiple melting/cooling cycles was examined. 1 mL samples were heated as in Example 4. After cooling to room temperature, the samples were melted again for up to five total cycles. After each heating cycle, viscosity was determined as described above. As shown in Table 3, the EX05 samples showed the same viscosity increases over the unheated control sample even after 5 heat cycles.

TABLE 3
Viscosity increase of EXO5 after heat cycling

| Melt cycles | % Viscosity increase over native EXO5 |
|---|---|
| 1 | 562 |
| 2 | 537 |
| 3 | 570 |
| 4 | 561 |
| 5 | 541 |

### Example 7. Gelation of Heat-Treated Exo5

Gels are often used to prepare food and personal care products. A water-soluble biopolymer that gelled upon cooling after a heat cycle would aid in preparation and liquid handling of such products. Further, a gel that melts at a lower temperature than ingredients such as agar would preserve the activity of heat sensitive ingredients. Other benefits could include efficiency of sterilization, and cold chain or shelf-life stability in providing resistance to temperature fluctuations during transit or storage.

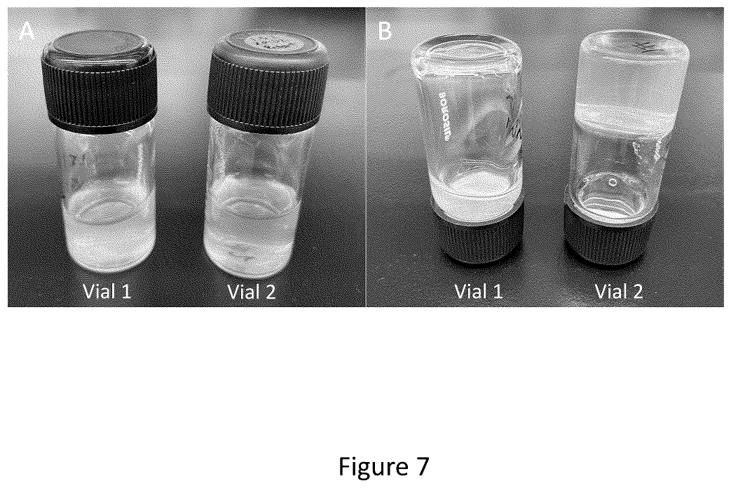

Example 4 describes the effect of a heat cycle on the viscosity of EXO4 and EXO5 at concentrations of 1% (w/v). To examine the effect of a high heat cycle on higher concentrations of EXO5, purified EXO5 was dissolved in water at 1.5% (w/v) and allowed to hydrate overnight at room temperature. FIG. 7 shows images of two 15 mL glass vials that were each filled with 10 mL of a 1.5% EXO5 solution. One vial (vial 1) was not heated. The other vial (vial 2) was autoclaved and cooled back to room temperature. Upon inversion of the glass vials (FIG. 7B), the heat-treated EXO5 solution formed a weak gel and was immobile relative to the control sample, which had not solidified. The heated sample displayed a zero-shear viscosity of 2865 Pa·s. As shown in this Example, EXO5 has the surprising property of gel formation after heat treatment and upon cooling.

### Example 8. Viscosity of EXO5 in a Model Body Wash

TABLE 4
Ingredients and their concentrations in a model body wash formula

| Ingredient | % Final volume |
|---|---|
| Exo5 | 1.5 |
| Sodium dodecyl sulfate (25%) | 25 |
| Decyl glucoside (50%) | 6 |
| Water | q.s. |

Body washes and gels benefit from high viscosity and shear thinning for application. Body washes and gels can be made from a combination of surfactants and water, and optionally preservatives, thickening agents, suspension aids, fragrance, chelants, colorants, conditioning agents, etc. To test the ability of EXO5 to provide viscosity and stabilize a model body wash formula, purified EXO5 was incorporated as an ingredient into the recipe listed above. Using techniques described above for measurement, EXO5 effectively viscosified (130 Pa·s at 0.01 s⁻¹) the model body wash and provided substantive texture. After use, skin is left with a clean, soft, feel. Next, the effect of heat-treated EXO5 on the model body wash was observed. Heat treating EXO5 before incorporation increased viscosity to 869 Pa·s at 0.01 s⁻¹. When the rheometer reached high shear rates, a smooth foam was formed, indicating that EXO5 can also be used for foaming soaps and washes.

### Example 9. Retort Treatment Using ExoS

Many shelf-stable foods and other products are prepared by retort treatment—sterilization under high temperature and pressure, similar to autoclaving. High viscosity reduces heat transfer, posing a problem for retort efficiency in thick products. EXO5's heat induced thickening would make it an ideal candidate for the preparation of shelf-stable foods and other products. Incorporating EXO5 into products at low concentration would produce a thin product more amenable to retort processing. As the product cools it would thicken to its desired final viscosity.

To test the feasibility of using EXO5 for retort treatment, a 1.5% solution of EXO5 in water was subjected to autoclaving for 20 min at 121° C. The treated solution had a viscosity 6.5× that of native EXO5 (1027 Pa·s compared to 157 Pa·s at 0.01 s⁻¹). Since high viscosity reduces heat transfer, a product with low initial viscosity that thickens after retorting would be beneficial to processing of shelf stable packaged food products, and other viscous products where heat sterilization is required.

1. A composition comprising a biopolymer, wherein the biopolymer is a succinoglycan lacking succinyl moieties, and wherein the biopolymer is heat stable and/or heat activated.

2. The composition of claim 1, wherein at least 50%, at least 55%, at least 60%, at least 65%, at least 70%, at least 75%, at least 80%, at least 85%, or at least 90% of the repeating units of the biopolymer comprise a pyruvyl moiety, and/or
wherein at least 30%, at least 35%, at least 40%, at least 45%, at least 50%, at least 55%, at least 60%, at least 65%, at least 70%, at least 75%, at least 80%, at least 85%, or at least 90% of the repeating units of the biopolymer comprise an acetyl moiety or wherein less than 30%, less than 25%, less than 20%, less than 15%, less than 10%, or less than 5% of the repeating units of the biopolymer comprise an acetyl moiety.

3. (canceled)

4. (canceled)

5. A composition comprising a biopolymer composed of repeating units of the structure: 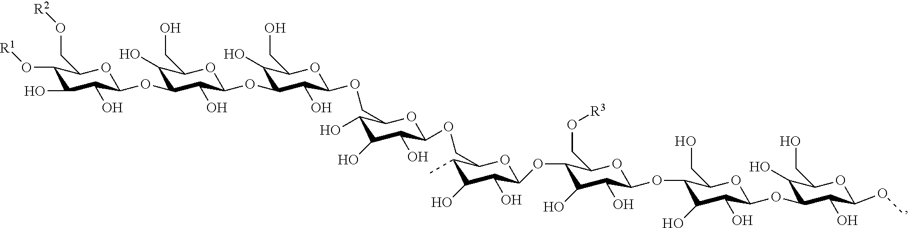
wherein the dotted lines indicate the connection between adjacent units; and
wherein for each unit, independently:
R³ is hydrogen or an acetyl moiety (C(O)CH₃);
R¹ and R² are each hydrogen, or are taken together to form a pyruvyl moiety (C(CH₃)CO₂H);
wherein at least 50%, at least 55%, at least 60%, at least 65%, at least 70%, at least 75%, at least 80%, at least 85%, or at least 90% of the units comprise a pyruvyl moiety and/or
wherein at least 30%, at least 35%, at least 40%, at least 45%, at least 50%, at least 55%, at least 60%, at least 65%, at least 70%, at least 75%, at least 80%, at least 85%, or at least 90% of the units comprise an acetyl moiety or wherein less than 30%, less than 25%, less than 20%, less than 15%, less than 10%, or less than 5% of the units comprise an acetyl moiety.

6. (canceled)

7. The composition of claim 5, wherein at least 30%, at least 35%, at least 40%, at least 45%, at least 50%, at least 55%, at least 60%, at least 65%, at least 70%, at least 75%, at least 80%, at least 85%, or at least 90% of the units have the structure:

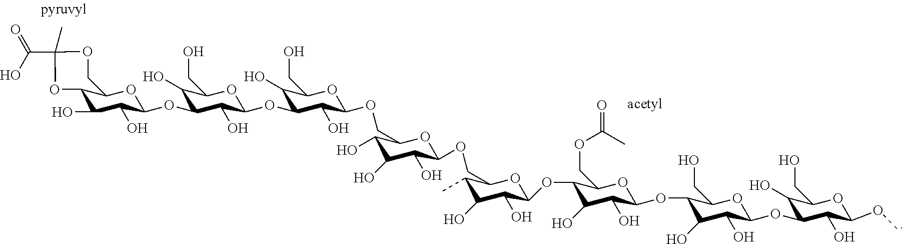

8. The composition of claim 1, wherein the composition is heat stable and/or heat activated when heated to at least 75° C., at least 80° C., at least 85° C., at least 90° C.,
optionally wherein the viscosity of the composition is at least 2-fold, at least 3-fold, at least 4-fold, at least 5-fold, or at least 6-fold higher after heating to a temperature of at least 75° C., at least 80° C., at least 85° C., at least 90° C., compared to the viscosity of the composition at the same temperature prior to heating, and/or wherein the viscosity of the composition is at least 2-fold, at least 3-fold, at least 4-fold, at least 5-fold, or at least 6-fold higher at a second temperature after heating than at a first temperature prior to heating, wherein the first temperature and the second temperature are the same and are between 0° C.-50° C., or 0° C.-40° C., or 0° C.-30° C., or 10° C.-50° C., or 10° C.-40° C., or 10° C.-30° C.

9. (canceled)

10. (canceled)

11. (canceled)

12. (canceled)

13. The composition of claim 5, wherein at least at least 50%, at least 55%, at least 60%, at least 65%, at least 70%, at least 75%, at least 80%, at least 85%, or at least 90% of the units have the structure:

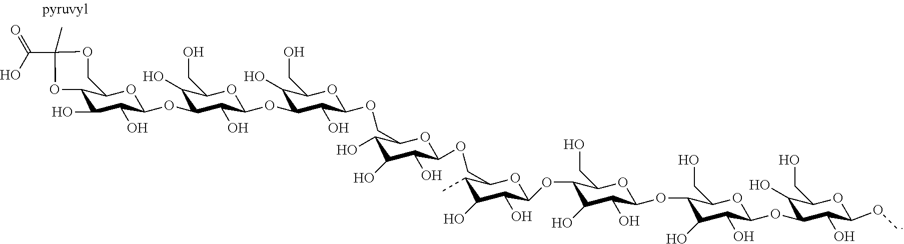

14. (canceled)

15. The composition of claim 1, wherein the viscosity of the composition is substantially the same before and after heating to a temperature of at least 75° C., at least 80° C., at least 85° C., at least 90° C., and/or wherein the viscosity of the composition is substantially the same at a first temperature before heating and at a second temperature after heating, wherein the first temperature and the second temperature are the same and are between 0° C.-50° C., or 0° C.-40° C., or 0° C.-30° C., or 10° C.-50° C., or 10° C.-40° C., or 10° C.-30° C.

16. (canceled)

17. The composition of claim 1, wherein the composition has been heated to at least 60° C., at least 70° C., at least 80° C., or at least 90° C. until the composition is uniformly heated and/or for at least 1 second, at least 5 seconds, at least 10 seconds, at least 30 seconds, at least 1 minute, at least 3 minutes, at least 5 minutes, at least 10 minutes, or at least 30 minutes.

18. The composition of claim 1, wherein the composition comprises 0.01 to 1%, or 0.1 to 2%, or 0.1-10% biopolymer.

19. The composition of claim 1, wherein the composition has been pasteurized, optionally by heating to a temperature of at least 80° C.

20. (canceled)

21. The composition of claim 1, which is a water-in-oil emulsion.

22. The composition of claim 1, wherein the composition is a food composition, optionally a packaged food, shelf-stable food, pressure treated food, food that undergoes heating prior to consumption or during transit, or a sterilizable food.

23. (canceled)

24. (canceled)

25. (canceled)

26. (canceled)

27. (canceled)

28. (canceled)

29. The composition of claim 1, wherein the composition is a cosmetic, personal care composition, or hair care product.

30. (canceled)

31. (canceled)

32. The composition of claim 1, wherein the composition comprises a surfactant.

33. The composition of claim 32, wherein the composition comprises one or more of an ionic, nonionic, amphoteric, anionic and/or zwitterionic surfactant,.

34. The composition of claim 1, wherein the composition is a home care or cleaning product.

35. The composition of claim 1, which is a drilling fluid, optionally wherein the composition comprises potassium and/or sodium salt and/or optionally wherein the viscosity of the composition is substantially restored after at least one, at least two, or at least three heating and cooling cycles.

36. (canceled)

37. (canceled)

38. (canceled)

39. (canceled)

40. The composition of claim 1, which is a gravel pack slurry, optionally wherein the gravel pack slurry comprises sand and water, wherein the biopolymer is present in an amount sufficient to maintain the sand in suspension in the water at 0° C.-50° C., or 0° C.-40° C., or 0° C.-30° C., or 10° C.-50° C., or 10° C.-40° C., or 10° C.-30° C.

41. (canceled)

42. (canceled)

43. A method comprising heating the composition of claim 1, optionally to at least 75° C., at least 80° C., at least 85° C., at least 90° C. until the composition is uniformly heated and/or for at least 1 second, 5 seconds, at least 10 seconds, at least 30 seconds, at least 1 minute, at least 3 minutes, at least 5 minutes, at least 10 minutes, or at least 30 minutes,
wherein the method further optionally comprises:
cooling the composition to a temperature between 0° C.-50° C., or 0° C.-40° C., or 0° C.-30° C., or 10° C.-50° C., or 10° C.-40° C., or 10° C.-30° C., after heating, and/or
more than one heating or cooling cycle, and/or
optionally wherein the viscosity of the composition is substantially the same after cooling as it was at the same temperature before heating, or wherein the viscosity of the composition is at least 2-fold, at least 3-fold, at least 4-fold, at least 5-fold, or at least 6-fold higher after cooling than at the same temperature before heating.

44. (canceled)

45. (canceled)

46. (canceled)

47. (canceled)

48. (canceled)

49. A method comprising applying pressure ≥100 MPa to the composition of claim 1, optionally wherein the viscosity of the composition is either substantially the same after application of pressure as it was before application of pressure, or the viscosity of the composition is at least 2-fold, at least 3-fold, at least 4-fold, at least 5-fold, or at least 6-fold higher after application of pressure than before application of pressure.

50. (canceled)

51. A method of preparing a composition comprising incorporating the composition of claim 1 into a food or beverage,
wherein the method optionally further comprises:
heating the composition, to at least 75° C., at least 80° C., at least 85° C., at least 90° C. until the composition is uniformly heated and/or for at least 1 second, at least 5 seconds, at least 10 seconds, at least 1 minute, at least 3 minutes, at least 5 minutes, at least 10 minutes, or at least 30 minutes, wherein after heating, optionally cooling the composition to a temperature between 0° C.-50° C., or 0° C.-40° C., or 0° C.-30° C., or 10° C.-50° C., or 10° C.-40° C., or 10° C.-30° C., wherein after heating, the viscosity of the composition is either substantially the same after cooling as it was at the same temperature before heating, or the viscosity of the composition is at least 2-fold, at least 3-fold, at least 4-fold, at least 5-fold, or at least 6-fold higher after cooling than at the same temperature before heating,
more than one heating and cooling cycle, and/or
applying pressure ≥100 MPa to the composition, optionally wherein the viscosity of the composition is either substantially the same after application of pressure as it was before application of pressure, or the viscosity of the composition is at least 2-fold, at least 3-fold, at least 4-fold, at least 5-fold, or at least 6-fold higher after application of pressure than before application of pressure.

52. A method of preparing a composition comprising incorporating the composition of claim 1 into a cosmetic, personal care composition, or hair care product,
wherein the method optionally further comprises:
heating the composition, to at least 75° C., at least 80° C., at least 85° C., at least 90° C. until the composition is uniformly heated and/or for at least 1 second, at least 5 seconds, at least 10 seconds, at least 1 minute, at least 3 minutes, at least 5 minutes, at least 10 minutes, or at least 30 minutes, wherein after heating, optionally cooling the composition to a temperature between 0° C.-50° C., or 0° C.-40° C., or 0° C.-30° C., or 10° C.-50° C., or 10° C.-40° C., or 10° C.-30° C., wherein after heating, the viscosity of the composition is either substantially the same after cooling as it was at the same temperature before heating, or the viscosity of the composition is at least 2-fold, at least 3-fold, at least 4-fold, at least 5-fold, or at least 6-fold higher after cooling than at the same temperature before heating,
more than one heating and cooling cycle, and/or
applying pressure ≥100 MPa to the composition, optionally wherein the viscosity of the composition is either substantially the same after application of pressure as it was before application of pressure, or the viscosity of the composition is at least 2-fold, at least 3-fold, at least 4-fold, at least 5-fold, or at least 6-fold higher after application of pressure than before application of pressure.

53. (canceled)

54. (canceled)

55. (canceled)

56. (canceled)

57. (canceled)

58. (canceled)

59. (canceled)

60. A method of maintaining or increasing the stability of a product exposed to at least one heating and cooling cycle comprising incorporating a composition of claim 1 into the product, optionally wherein the heating and cooling cycle comprises heating the product to at least 75° C., at least 80° C., at least 85° C., at least 90° C. until the composition is uniformly heated and/or for at least 1 second, at least 5 seconds, at least 10 seconds, at least 30 seconds, at least 1 minute, at least 3 minutes, at least 5 minutes, at least 10 minutes or at least 30 minutes, and after heating, cooling the product to a temperature between 0° C.-50° C., or 0° C.-40° C., or 0° C.-30° C., or 10° C.-50° C., or 10° C.-40° C., or 10° C.-30° C., and/or optionally wherein after heating, the viscosity of the product is either substantially the same after cooling as it was at the same temperature before heating, or the viscosity of the product is at least 2-fold, at least 3-fold, at least 4-fold, at least 5-fold, or at least 6-fold higher after cooling than at the same temperature before heating.

61.-69. (canceled)
